(19) United States
(12) Patent Application Publication
(10) Pub. No.: US 2009/0227984 A1
(43) Pub. Date: Sep. 10, 2009

(54) System and methods of intubation

(76) Inventor: Robert T. Kizer, Omaha, NE (US)

Correspondence Address: Valauskas & Pine LLC, 150 South Wacker Drive, Suite 620, Chicago, IL, 60606, US

(21) Appl. No.: 12/380,005

(22) Filed: Feb. 20, 2009

Related U.S. Application Data
(60) Provisional application No. 61/066,703, filed on Feb. 21, 2008.

(51) Int. Cl. A61M 25/01; A61J 15/00

(52) U.S. Cl. 604/528; 604/516

(57) ABSTRACT

The present invention includes various devices for improved nasogastric intubation to ensure proper placement of the device within the body cavity as well as minimize trauma and achieve patient comfort both during and after an intubation procedure.

### FIELD OF THE INVENTION

The present invention generally relates to a medical system and methods and, more particularly, to a system and methods for nasogastric intubation.

The present invention is discussed in the following largely with reference to nasogastric intubation, but the present invention may be applicable to a variety of intubation procedures, for example, orogastric or orotracheal.

### BACKGROUND OF THE INVENTION

Nasogastric or nasotracheal intubation is a medical procedure involving the insertion of a tube, referred to herein as a nasogastric tube (“NG tube”), into a body cavity, such as through the nose, past the phyarnx, down through the esophagus and into the stomach.

Before a NG tube is inserted into the body cavity, a health care provider, such as a nurse or doctor, typically measures the distance from the tip of the patient's nose, to their ear and down to the xyphoid process. The NG tube is then marked at this level to ensure that the tube has been inserted far enough into the patient's stomach. The conventional insertion process begins with the first lubrication of the end of the NG tube, then its insertion into and through one of the patient's nostrils. The NG tube is then moved through the nasal cavity and down into the throat. Once the NG tube is past the pharynx, it is rapidly inserted down into the esophagus and stomach.

During insertion, great care must be taken to ensure that the NG tube is positioned properly and that it has not passed through the windpipe and down into the lungs. To ensure proper placement, it is a known method to inject air into the tube; if the air is heard in the stomach with a stethoscope, then the NG tube is in the correct position. Another method to ensure proper placement of the NG tube is to aspirate fluid from the tube with a syringe. This fluid is then tested with pH paper to determine the acidity of the fluid. If the pH is below a certain level, such as 5.5 pH, then the NG tube is in the correct position. Otherwise, verification of tube position may be obtained with an X-ray of the chest/abdomen.

The main use of a NG tube is for feeding and the administration of oral agents, such as drugs. Another use of a NG tube is for nasogastric aspiration, or suction, to drain contents from the stomach. Nasogastric aspiration is mainly used to remove gastric secretions and swallowed air in a patient with gastrointestinal obstructions. Nasogastric aspiration can also be used when a potentially toxic liquid, such as poison, has been ingested. Nasogastic aspiration is also used for preparation before surgery under anesthesia, and to extract samples of gastric liquid for analysis.

Nasogastric intubation may cause trauma such as nose bleeds, sinusitis, and a sore throat. More significant trauma may occur including erosion of the nose where the tube is anchored, esophageal perforation, pulmonary aspiration, a collapsed lung, or intracranial placement of the NG tube.

Overall, it is recognized that the currently available instruments to facilitate nasogastric intubation are not without serious shortcomings. Accordingly, there is a need for improved nasogastric intubation to ensure proper placement of the device within the body cavity as well as to minimize trauma and to achieve greater patient comfort both during and after an intubation procedure.

### SUMMARY OF THE INVENTION

The present invention is directed to various embodiments by which nasogastric intubation can be facilitated. With the use of these embodiments, nasogastric intubation may be quick and safe: proper placement of the NG tube may be ensured, complications minimized, and patient comfort achieved both during and after an intubation procedure.

The present invention is discussed in the following in reference to nasogastric intubation, but the present invention is applicable to a variety of intubation procedures, for example, orogastric or orotracheal.

As shown in FIG. 1, the pharynx 100, which is the part of the neck and throat, is situated immediately posterior to the oral cavity or mouth 102 and nasal cavity 104. The pharynx 100 includes a nasopharynx 106, oropharynx 108 and hypopharynx 110. The nasopharynx 106 lies behind the nasal cavity 104 and typically extends from the hard and soft palates to the base of the skull. The oropharynx 108 lies behind the oral cavity 102. The hypopharynx 110, sometimes called the laryngopharynx, extends to the larynx 112, which is situated just below the junction that diverges into the trachea 114 and esophagus 116. The esophagus 116 leads to the stomach, or abdomen. For purposes of this application, “pharynx” refers to the nasophayrnx, oropharynx, hypopharynx and sometimes larynx.

The present invention includes devices to properly position a nasogastric tube (“NG tube”) through a nostril of the nose, past the pharynx, down through the esophagus and into the stomach.

One embodiment the present invention is a slider device that includes a collapsed state and an erected state. The slider device may be inserted into the pharynx in a collapsed state. The slider device achieves an erected state upon an instrument, such as a NG tube or other instruments, threaded therethrough.

Another embodiment of the present invention is a stylet device with a pellet element positioned on one end thereof. The pellet element may be weighted to assist in positioning the stylet device within the pharynx. The stylet device guides instruments, such as a NG tube or other instruments, into the pharynx by allowing the instruments to be threaded over the stylet device or following alongside the stylet device.

Yet another embodiment of the present invention is a director device. The director device includes a guide element that may be manipulated by a user to curve or angle one end or a portion of the director device for proper positioning within the pharynx. Upon proper positioning of the director device, an instrument is inserted therethrough.

Yet another embodiment of the present invention is a swallower device that includes one or more bladder elements. The bladder element achieves an inflated state from a deflated state when injected with a substance. The bladder element anchors the instrument prior to insertion within the pharynx as well as simulates a food bolus to assist the patient in advancing the swallower device, including instruments, into his or her stomach.

For purposes of this application, the term “instrument” includes not only a NG tube, but also a slider device, stylet device, swallower device, and director device. Thus, the devices according to the present invention may be used in conjunction with one another for insertion into the nostril and in the pharynx of a patient so that a NG tube can be positioned through a nostril of the nose, past the pharynx, down through the esophagus and into the stomach.

For example, in one embodiment the director device may be used with the swallower device to insert and position a NG tube. The director device may be inserted into the nostril and in the pharynx of a patient. The NG tube may be positioned on the swallower device, which is then threaded within the director device.

In another embodiment, the slider device may be used with the swallower device. The slider device may be inserted into the nostril and in the pharynx of a patient. The NG tube may be positioned on the swallower device, which is then threaded within the slider device, thereby erecting the slider device.

In yet another embodiment, the slider device may be used with the director device. The slider device may be inserted into the nostril and in the pharynx of a patient. The director device may be threaded through the slider device, thereby erecting the slider device, and properly positioned within the pharynx. The NG tube may be then inserted in the director device.

In another embodiment, the stylet device may be used with the director device. The director device may be inserted into the nostril and in the pharynx of a patient. The NG tube may be positioned on the stylet device, which is then threaded within the director device.

In another embodiment, the stylet device may be used with the swallower device. The stylet device is inserted into the nostril and in the pharynx of a patient. The NG tube is positioned on the swallower device, which is then threaded over the stylet device, thereby erecting the slider device. Once positioned, the stylet device may be removed from the NG tube. This embodiment is contemplated for use with larger diameter NG tubes.

In another embodiment, the stylet device may be used with the slider device. The slider device may be inserted into the nostril and in the pharynx of a patient. The NG tube may be positioned on the stylet device, which is then threaded within the slider device, thereby erecting the slider device.

In yet another embodiment, the slider device, director device and swallower device may all be used in combination. The slider device may be inserted into the nostril and in the pharynx of a patient. The director device may be threaded through the slider device, thereby erecting the slider device, and inserted into the pharynx. The NG tube may be positioned on the swallower device, which is then threaded within the erected slider device including director device.

In yet another embodiment, the slider device, director device and stylet device may all be used in combination. The slider device may be inserted into the nostril and in the pharynx of a patient. The director device may be threaded through the slider device, thereby erecting the slider device, and properly positioned within the pharynx. The NG tube may be positioned on the stylet device, which is then threaded within the slider device including director device.

In yet another embodiment, the stylet device, slider device and swallower device may all be used in combination. The stylet device is inserted into the nostril and in the pharynx of a patient. The slider device is threaded over the stylet device thereby erecting the slider device. Once positioned, the stylet device may be removed. The NG tube is positioned on the swallower device and inserted through the erected slider device.

In yet another embodiment, the stylet device, slider device, director device and swallower device may all be used in combination. The stylet device is inserted into the nostril and in the pharynx of a patient. The slider device is threaded over the stylet device, thereby erecting the slider device. Once positioned, the stylet device may be removed. The director device is inserted through the erected slider device. The swallower device with the NG tube positioned thereon, may then be inserted into the director device. Once positioned, the director device may be removed.

The present invention and its attributes and advantages will be further understood and appreciated with reference to the detailed description below of presently contemplated embodiments, taken in conjunction with the accompanying drawings.

### BRIEF DESCRIPTION OF THE DRAWINGS

FIG. 1 is a cross-sectional view of a pharynx;

FIG. 2A is a perspective view of a slider device in a collapsed state according to the present invention;

FIG. 2B is a perspective view of a slider device in an erected state according to the present invention;

FIG. 3 is a perspective view of a stylet device according to the present invention;

FIG. 4 is a perspective view of a director device according to the present invention;

FIG. 5A is a perspective view of a swallower device with deflated bladders according to the present invention; and

FIG. 5B is a perspective view of a swallower device with inflated bladders according to the present invention.

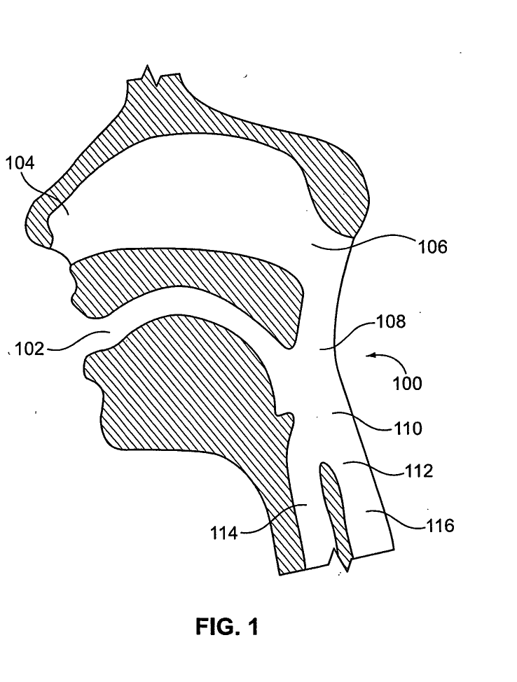

### DETAILED DESCRIPTION OF EMBODIMENTS OF THE INVENTION

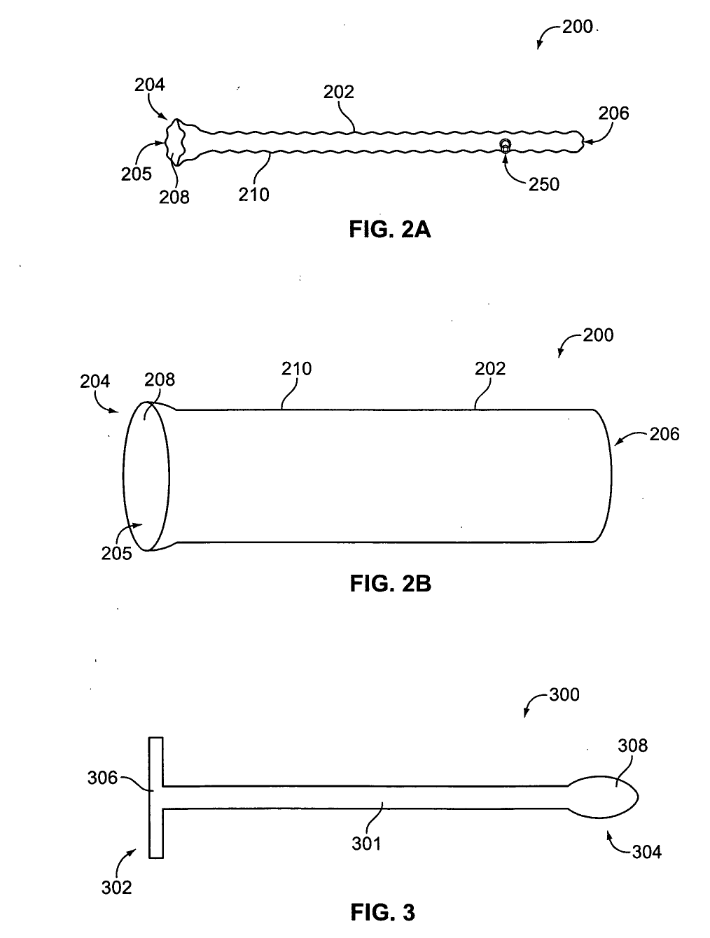

The slider device 200, as shown in FIGS. 2A and 2B, includes a body component 202 extending from a first end 204 to a second end 206. The body component 202 is cylindrical in shape with an inside surface 208 and an outside surface 210, but any shape that can be inserted into the nostril and in the pharynx of a patient is contemplated. The slider device 200 may be made from any flexible material, such as plastic or rubber. The flexible material may further include porous properties to allow nasal secretions to lubricate the slider device 200. It is also contemplated that the flexible material may also include a water activated lubricant. Lubrication of the slider device 200 assists in inserting the device into the nostril and into the pharynx of a patient.

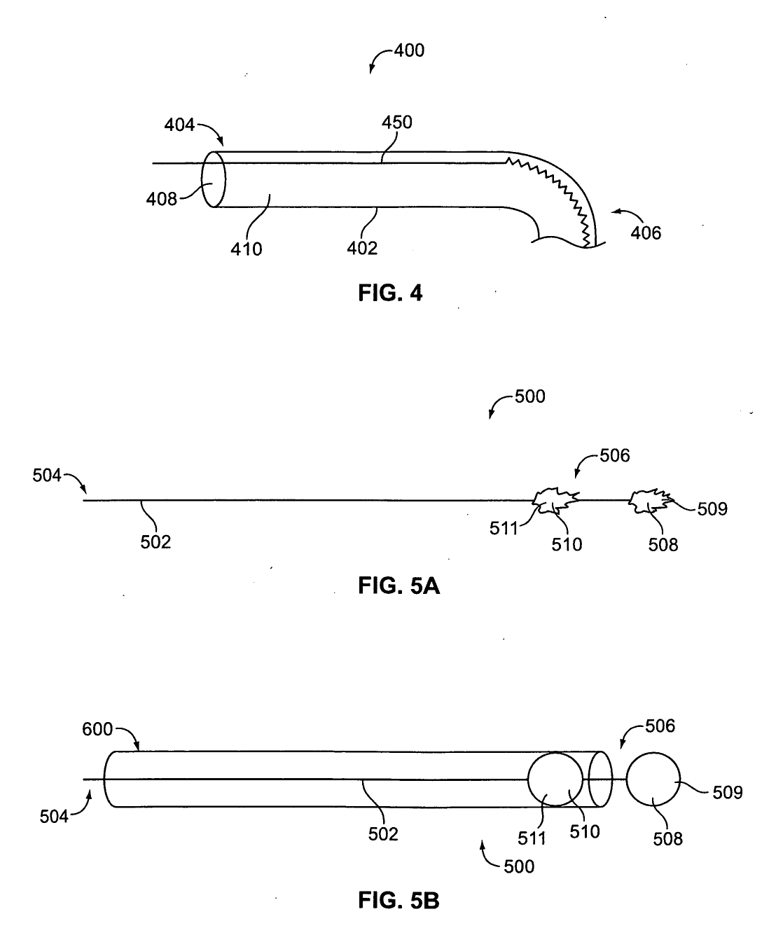

The second end 206 of the slider device 200 is inserted into the nostril of a patient and in the pharynx prior to insertion of the NG tube or other instrument. In one embodiment, the slider device 200 may be used in conjunction with the stylet device 300 discussed more fully in reference to FIG. 3. In another embodiment, the slider device 200 may be used in conjunction with the director device 400 discussed more fully in reference to FIG. 4 and yet in another embodiment, the slider device 200 may be used in conjunction with the swallower device 500 discussed more fully in reference to FIG. 5.

The first end 204 may either be completely inserted into the nostril or may be exposed outside the nostril. The slider device 200 is inserted into the patient in a collapsed state, as shown in FIG. 2A. Upon positioning within the pharynx, the slider device 200 achieves an erected state as shown in FIG. 2B.

In one embodiment, the slider device 200 is erected by threading an instrument, for example, a NG tube, stylet device, swallower device, and director device, through the first end 204, within the inside surface 208 of the body component 202, and through the second end 206. In another embodiment, the first end 204 includes a lumen end 205 that maintains an open position to facilitate the insertion of an instrument into the collapsed state of the slider device 200. The lumen end 205 is desirable in embodiments wherein the first end 204 of the slider device 200 is exposed, such as when a patient has a deviated septum within the nose or constricted areas within the pharynx.

In other embodiments, the slider device 200 includes a weak adhesion element 250 on the inside surface 208 to maintain the collapsed state shown in FIG. 2A. When an instrument is threaded through the slider device 200, the weak adhesion element 250 breaks to achieve the erected state shown in FIG. 2B. The weak adhesion element 250 may be an adhesive or spot weld of a portion of the inside surface 208 together with another portion of the inside surface 208 of the body component 202.

The stylet device 300, as shown in FIG. 3, includes a shaft component 301 extending from a near end 302 to a far end 304. The shaft component 301 is cylindrical in shape, but any small cross-sectional shape that can be inserted into the nostril and in the pharynx of a patient is contemplated. The shaft component 301 of the stylet device 300 may be made from any flexible material, such as plastic or rubber.

The near end 302 includes a handle element 306. In one embodiment, the far end 304 of the stylet device 300 includes a pellet element 308. The pellet element 308 may be made from any flexible material, such as plastic or rubber. The pellet element 308 may be spherical in shape, but any shape is contemplated that may be inserted into the nostril and in the pharynx of a patient. Further, the pellet element 308 may be weighted to assist in location of the stylet device 300.

The far end 304 of the stylet device 300 is inserted into the nostril and in the pharynx of a patient such that the near end 302 is exposed outside the nostril. Instruments such as a NG tube, slider device, swallower device, and director device, are inserted into the nostril and in the pharynx of a patient using the stylet device 300 as a guide. For example, a NG tube can be guided by the stylet device 300 by engulfing the shaft component 301 prior to insertion into the nostril and in the pharynx of a patient. As another example, the stylet device 300 may be inserted into the nostril and in the pharynx of a patient and then the NG tube is inserted into the nostril and in the pharynx to follow alongside the shaft component 301. In another embodiment, the stylet device 300 may be used in conjunction with the director device 400 discussed more fully in reference to FIG. 4. In another embodiment, the stylet device 300 may be used in conjunction with the swallower device 500 discussed more fully in reference to FIG. 5.

The director device 400, as shown in FIG. 4, includes a tubular component 402 extending from a proximal end 404 to a distal end 406. The tubular component 402 is cylindrical in shape with an interior surface 408 and an exterior surface 410, but any shape that can be inserted into the nostril and in the pharynx of a patient is contemplated. The tubular component 402 of the director device 400 may be made from any flexible material, such as plastic or rubber.

The proximal end 404 includes a guide element 450 made from any flexible material, such as metal, plastic or rubber, that extends through the interior surface 408 and attaches at the distal end 406. The guide element 450 attaches to the interior surface 408 of the tubular component 402 by any locking means known to those skilled in the art, for example, adhesive.

The distal end 406 of the director device 400 is inserted into the nostril and in the pharynx of a patient such that the proximal end 404 is exposed outside the nostril. The guide element 450 is then manipulated, for example to curve or angle the distal end 406 downward towards and past the pharynx, specifically the oropharynx.

Upon positioning the director device 400, instruments such as a NG tube, slider device, swallower device, and director device, are inserted through the interior surface 408. In one embodiment, the director device 400 may be used in conjunction with the swallower device 500 discussed more fully in reference to FIG. 5. In another embodiment, the director device 400 may be used in conjunction with the slider device 200 along with the stylet device 300.

The swallower device 500, as shown in FIGS. 5A and 5B, includes a conduit component 502 extending from a bottom end 504 to a tip end 506. The conduit component 502 is cylindrical in shape, but any shape that can be inserted into the nostril and in the pharynx of a patient is contemplated. The conduit component 502 of the swallower device 500 may be made from any flexible material, such as plastic or rubber.

The tip end 506 includes a first bladder element 508 and a second bladder element 510. Each bladder element 508, 510 is made from any flexible material, such as plastic or rubber. The first bladder element 508 includes a first cavity 509 and the second bladder element 510 includes a second cavity 511. The bladder elements 508, 510 inflate as shown in FIG. 5B when injected with a substance, such as air, saline or water. The bladder elements 508, 510 may be injected with a substance, for example, via a syringe inserted into the bottom end 504 of the conduit component 502.

Prior to injection, the bladder elements 508, 510 are deflated as shown in FIG. 5A to position an instrument, such as a NG tube, slider device, stylet device, and director device, over the conduit component 502 such that the first bladder 508 extends outside the instrument and the second bladder 510 remains inside the instrument as shown in FIG. 5B, wherein the instrument is a NG tube 600. After positioning of the NG tube 600 about the swallower device 500, the second cavity 511 of the second bladder element 510 is inflated to anchor the NG tube 600. The tip end 506 of the swallower device 500 is inserted into the nostril and in the pharynx of a patient. Upon the tip end 506 located beyond the nasopharynx and into the oropharynx, the first cavity 509 of the first bladder element 508 is inflated to provide the patient with the sensation that a food bolus is in his or her pharynx to assist the patient in advancing the swallower device including NG tube into his or her stomach.

Once the swallower device 500 including instrument are located within the stomach, the first cavity 509 of the first bladder element 508 and second cavity 511 of the second bladder element 510 are deflated such as by retracting the substance via the syringe that is inserted into the bottom end 504 of the conduit housing 502 so that the swallower device 500 can be removed while allowing the instrument to remain.

In one embodiment, the swallower device 500 may be used in conjunction with the director device 400. In another embodiment, the swallower device 500 may be used in conjunction with the slider device 200 along with the director device 400. In another embodiment, the swallower device 500 may be used in conjunction with the slider device 200 along with the stylet device 300. In yet another embodiment, the swallower device 500 may be used in conjunction with the slider device 200 along with the stylet device 300 along with the director device 400.

While the disclosure is susceptible to various modifications and alternative forms, specific exemplary embodiments thereof have been shown by way of example in the drawings and have herein been described in detail. It should be understood, however, that there is no intent to limit the disclosure to the particular embodiments disclosed, but on the contrary, the intention is to cover all modifications, equivalents, and alternatives falling within the scope of the disclosure as defined by the appended claims.

What is claimed is:

1. A slider device for intubation, comprising:
a body component extending from a first end to a second end including an inside surface and an outside surface, wherein said body component achieves an erected state from a collapsed state by inserting an instrument within said first end and through said inside surface.

2. The slider device of claim 1 further comprising a weak adhesion element on said inside surface of said body component.

3. The slider device of claim 1, wherein said first end includes a lumen end.

4. The slider device of claim 1, wherein said instrument is a stylet device.

5. The slider device of claim 1, wherein said instrument is a swallower device.

6. The slider device of claim 1, wherein said instrument is a director device.

7. A stylet device for intubation, comprising:
a shaft component extending from a near end to a far end;
a handle element positioned on said near end; and
a pellet element positioned on said far end, wherein said pellet element is located by manipulating said handle element.

8. The stylet device of claim 7, wherein said pellet element is weighted.

9. A director device for intubation, comprising:
a tubular component extending from a proximal end to a distal end including an interior surface and an exterior surface;
a guide element positioned on said proximal end, wherein said guide element extends through said interior surface and attaches to said distal end, and said distal end is located by manipulating said guide element.

10. The director device of claim 9 further comprising a slider device inserted through said interior surface of said tubular component.

11. The director device of claim 9 further comprising a swallower device inserted through said interior surface of said tubular component.

12. The director device of claim 9 further comprising a director device inserted through said interior surface of said tubular component.

13. The director device of claim 10 further comprising a stylet device inserted through said interior surface of said tubular component.

14. A swallower device for intubation, comprising:
a conduit component extending from a bottom end to a tip end;
a first bladder element positioned on said tip end, wherein said first bladder element includes a first cavity;
a second bladder element positioned on said tip end, wherein said second bladder element includes a second cavity, and said first cavity of said first bladder element and said second cavity of said second bladder element each achieve an inflated state from a deflated state by injecting said cavities with a substance.

15. The swallower device of claim 14, wherein said substance is injected using a syringe inserted into said bottom end of said conduit component.

16. The swallower device of claim 14 further comprising an instrument positioned over said conduit component such that said second cavity of said second bladder element is inflated to anchor said instrument.

17. The swallower device of claim 14, wherein said first cavity of said first bladder element and said second cavity of said second bladder element achieve said deflated state from said inflated state by retracting said substance from said cavities.
